(19) United States
(12) Patent Application Publication
(10) Pub. No.: US 2013/0095504 A1
(43) Pub. Date: Apr. 18, 2013

(54) DISEASE MARKER DETECTION KIT AND DISEASE MARKER DETECTION METHOD

(71) Applicants: Tae Geol Lee, Daejeon (KR); Young-Lai Cho, Daejeon (KR); Mi Young Son, Jeonllabuk-do (KR)

(72) Inventors: Tae Geol Lee, Daejeon (KR); Young-Lai Cho, Daejeon (KR); Mi Young Son, Jeonllabuk-do (KR)

(73) Assignee: KOREA RESEARCH INSTITUTE OF STANDARDS AND SCIENCE, Daejeon (KR)

(21) Appl. No.: 13/806,203

(22) PCT Filed: Jun. 24, 2011
(86) PCT No.: PCT/KR2011/004610

§ 371 Date: Dec. 21, 2012

(30) Foreign Application Priority Data
Jun. 24, 2010 (KR) 10-2010-0059834

(51) Int. Cl. G01N 33/574

(52) U.S. Cl. CPC G01N 33/57488
USPC 435/7.4; 422/430

(57) ABSTRACT

Provided is a detection kit, which a disease detection kit used together with a secondary ion mass spectrometer in order to detect a disease marker contained in a biological sample, the detection kit including: a base including a noble metal thin film formed thereon; a reactant containing peptide specifically reacting with the disease marker; a first storage unit filled with the reactant; a test substance containing a biological sample of a possible disease carrier; a second storage unit filled with the test substance; a mixing unit mixing the reactant and the test substance with each other to prepare a detection substance containing the specific reactant, which is the peptide specifically reacted with the disease marker contained in the biological sample; and a contact unit contacting the detection substance prepared by the mixing unit with the base to bond the specific reactant to the noble metal film of the base.

### CROSS-REFERENCE TO RELATED APPLICATIONS

This application claims priority under 35 U.S.C. §119 to Korean Patent Application No. 10-2010-0059834, filed on Jun. 24, 2010, in the Korean Intellectual Property Office, the disclosure of which is incorporated herein by reference in its entirety.

### TECHNICAL FIELD

The following disclosure relates to a disease marker detection kit detecting a kind and a content of a disease marker capable of determining presence of a disease without marker materials, and a detection method of a disease marker. The detection kit and detection method according to the present invention may be used for an initial diagnosis, prognosis, disease monitoring, and the like.

### BACKGROUND

As it has recently been investigated that malignant neoplasm (cancer) is a main cause of death in Korean, a need for early diagnosis of cancer and development of an accurate diagnosis method has increased.

Early diagnosis of cancer was based on external changes of biological tissue according to growth of cancer cells. However, recently, a diagnosis and detection using bio-molecules existing at a trace concentration in biological tissue or cell such as blood, sugar chain, DNA, or the like, has been attempted (Korean Patent Laid-Open Publication No. 2008-0003472, International Patent Laid-Open Publication No. 2000-004149, US Patent Laid-Open Publication No. 2004-586856, and US Patent Laid-Open Publication No. 2003-690880).

However, the most generally used diagnosis method for cancer is a diagnosis method using a tissue sample obtained by biopsy or an image.

However, the biopsy may cause great pain in a patient, be expensive, and take a long time to diagnose the disease. In addition, in the case in which the patient actually has cancer, there is a risk of inducing cancer metastasis during a biopsy process, and in the case of a region at which tissue samples through the biopsy cannot be obtained, it may be impossible to diagnose the disease before a suspected tissue sample is extracted through a surgical operation.

In the diagnosis using images, cancer is diagnosed based on images obtained through an X-ray, nuclear magnetic resonance imaging method using a contrast medium attached with disease target substance, nuclear imaging, or the like. However, this diagnosis method using the image has disadvantages in that a possibility of misdiagnosis may be large according to a skill level of an operator or a reader, and the diagnosis is excessively dependent on the precision of an equipment providing the image. Further, even in the case of the most precise device, it is impossible to detect a tumor having a size of several mm or less, such that it may be difficult to detect the tumor at an early stage. In addition, since a patient or a possible disease carrier is exposed to a high energy electromagnetic wave generating mutation of genes, another disease may be induced, and a frequency of diagnosis through images may be limited.

In the case of gastrointestinal cancer, generally, presence or absence of the disease is diagnosed by observing the image through an endoscope with the naked eye, but the process may be significantly painful to the patient. In addition, although abnormality is discovered by observing the image with the naked eye, the biopsy should be essentially performed in order to accurately determine diseases such as malignant/benign tumor, polyp, or the like.

As an example of the diagnosis and detection using bio-molecules such as blood, sugar chain, DNA, or the like, that is present at a trace concentration in biological tissue or cell, there is detection of cell wall lytic enzymes.

Before proliferation, invasion, and metastasis of cancer cells occur, first, a damage to a basement membrane essentially occurs, and before invasion of the cancer cells into blood vessels, a sub-endothelial base membrane consisting of type IV collagen are degraded, first. It is known that matrix metalloproteinases (MMPs) are involved in this protein degradation process. Recently, at least twenty one of MMPs are known, and each of the MMPs has a different mass and various substrate-specific sites.

As the MMPs are highly expressed in cancer cells or cancer tissue of almost everyone, measurement of an amount of MMPs in human blood serum is used as a diagnostic marker for cancer (Lein, M. 1997, Clinic. Biochem., 491-496; Nikkola, J. 2005, Clin. Cancer Res., 5158-5166).

Generally, as a method of measuring activity of the MMPs, gelatin-substrate-zymography for measuring activity of gelatinase is used. This method, which is a method of measuring the activity of the MMPs at optical absorbance of each MMPs mass band according to the presence or absence of enzymes after gel staining using a gel including matrix protein, requires a large amount of enzymes, and experimental errors and a problem of reproducibility may be caused by performing the experiment through several steps.

In addition, the activity of the MMPs is measured by a method using antigen-antibody interaction in a solution such as an enzyme-linked immunosorbent assay (ELISA). However, in this method, fluorescent or luminescent labeling material as well as the antigen-antibody reaction are used, but experimental errors generated due to labeling (protein deformation generated by labeling, an error due to instability of label itself, and the like) are not solved yet.

### SUMMARY

An embodiment of the present invention is directed to providing a detection kit capable of non-invasively detecting a kind and a content of a disease marker used to determine presence or absence of a disease of a possible disease carrier without labeling; simply, rapidly, accurately, and reproducibly detecting a content of a disease marker; and being mass-produced with low cost.

Another embodiment of the present invention is directed to providing a detection method of a content of a non-invasive and unlabeled disease marker used to determine presence or absence of a disease of a possible disease carrier, and a detection method of a content of a disease marker capable of simply, accurately, and reproducibly detecting the content of the disease marker in a short time.

In one general aspect, there is provided a disease detection kit used together with a secondary ion mass spectrometer in order to detect a disease marker contained in a biological sample, the disease detection kit including: a base including a noble metal thin film formed thereon; a reactant containing peptide specifically reacting with the disease marker; a first storage unit filled with the reactant; a test substance containing a biological sample of a possible disease carrier; and a second storage unit filled with the test substance.

The disease detection kit may further include: a mixing unit mixing the reactant and the test substance with each other to prepare a detection substance containing the specific reactant, which is the peptide specifically reacted with the disease marker contained in the biological sample; and a contact unit contacting the detection substance prepared by the mixing unit with the base to bond the specific reactant to the noble metal film of the base.

The disease detection kit may further include: a storage part storing a test spectrum, which is a secondary ion mass spectrum of a test sample in which the specific reactant of the detection substance is bonded to the noble metal thin film; and a determining part determining a kind and a content of the disease marker contained in the biological sample based on at least one factor selected from the secondary ion mass and intensity of a peak present in the test spectrum stored in the storage part.

The disease marker may be a marker for cancer and a cell wall lytic enzyme. In addition, the cell wall lytic enzyme may be at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

In order to specifically react with the cell wall lytic enzyme, the peptide may be a substrate having a specific amino acid site for the cell wall lytic enzyme. In order to allow the specific reactant, which is the peptide specifically reacted with the cell wall lytic enzyme, to be maximally uniformly bonded to the noble metal thin film of the board in a monolayer form, an amino acid of an amine terminal or a carboxyl terminal of the peptide may be cysteine.

The noble metal thin film may be a gold thin film.

The biological sample may be feces, urine, tears, saliva, external secretions of the skin, external secretions of a respiratory tract, external secretions of enteric track, external secretions of the digestive tract, plasma, serum, blood, spinal fluid, lymph fluid, body fluids, tissue, tissue homogenates, part of tissue, cells, cell extracts, or in vitro cell culture.

In another general aspect, there is provided a method of detecting a disease marker in order to provide information required for diagnosing a disease, the detection method of a disease marker including: a) mixing a reactant containing a peptide specifically reacting with the disease marker with a test substance containing a biological sample of the possible disease carrier to prepare a detection substance containing a specific reactant, which is a peptide specifically reacted with the disease marker contained in the biological sample; b) contacting the detection material with the base including a noble metal thin film formed thereon to bond unreacted peptide and the specific reactant to the noble metal thin film; c) performing a secondary ion mass spectroscopy (SIMS) on the base contacted with the detection substance to obtain a test spectrum; and d) detecting a kind and a content of the disease marker based on at least one factor selected from a secondary ion mass and intensity of a peak present in the test spectrum.

The detection method of a disease marker may further include precipitating the disease marker contained in the biological sample using an immunoprecipitation method before step a), wherein the test substance includes a precipitation substance precipitated by the immunoprecipitation method.

The disease marker may be a marker for cancer and a cell wall lytic enzyme, wherein the cell wall lytic enzyme is at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

The peptide may be an unlabeled substrate having an amino acid site specifically reacting with the cell wall lytic enzyme. In addition, the peptide may be an unlabeled substrate having an amino acid site specifically reacting with two or more cell wall lytic enzymes different from each other.

More particularly, the peptide may be an unlabeled substrate having amino acid sites (specific amino acid sites) specifically reacting with each of the above-mentioned lytic enzymes and include an unlabeled substrate having an amino acid site specifically reacting with two or more cell wall lytic enzymes different from each other.

In order to allow the peptide specifically reacted with the cell wall lytic enzyme to be maximally uniformly bonded to the noble metal thin film of the board in a monolayer form, an amino acid of an amine terminal or a carboxyl terminal of the peptide may be cysteine.

The noble metal thin film may be a gold thin film.

The biological sample may be feces, urine, tears, saliva, external secretions of the skin, external secretions of a respiratory tract, external secretions of enteric track, external secretions of the digestive tract, plasma, serum, blood, spinal fluid, lymph fluid, body fluids, tissue, tissue homogenates, part of tissue, cells, cell extracts, or in vitro cell culture.

Other features and aspects will be apparent from the following detailed description, the drawings, and the claims.

### BRIEF DESCRIPTION OF THE DRAWINGS

FIGS. 1A to 1C are views showing examples of a base on which a noble metal thin film included in a detection kit according to the present invention is formed.

FIG. 2 is a view showing an example of a first storage unit provided in the detection kit according to the present invention.

FIG. 3 is a view showing results obtained by detecting activity of MMP-9 using urine as a biological sample and using a matrix having a MMP-9 specific sequence.

FIG. 4 is a view showing results obtained by detecting activity of MMP-9 using urine as a biological sample and using a general substrate of MMP.

### DETAILED DESCRIPTION OF EMBODIMENTS

Hereinafter, a manufacturing method according to present invention will be described in detail with reference to the accompanying drawings. The drawings to be provided below are provided by way of example so that the idea of the present invention can be sufficiently transported to those skilled in the art. Therefore, the present invention is not limited to the drawings to be provided below, but may be modified in many different forms. In addition, the drawings to be provided below may be exaggerated in order to clarify the scope of the present invention. Further, like reference numerals denote like elements throughout the specification.

Here, technical terms and scientific terms used in the present specification have the general meaning understood by those skilled in the art to which the present invention pertains unless otherwise defined, and a description for the known function and configuration obscuring the present invention will be omitted in the following description and the accompanying drawings.

A disease detection kit according to the present invention is a disease detection kit used together with a secondary ion mass spectrometer in order to detect a disease marker contained in a biological sample. The detection kit includes a base on which a noble metal film is formed, a reactant containing a peptide specifically reacting with the disease marker; a first storage unit in which the reactant is sealed and stored; a test substance containing the biological sample of a possible disease carrier; and a second storage unit in which the test substance is sealed and stored.

As the base of the disease detection kit, which is to serve to support the noble metal thin film and provide a space on which the noble metal thin film is formed, any material that does not affect analysis results at the time of SIMS analysis and chemically react with the reactant and the test substance but has excellent coupling strength with the noble metal thin film may be used as a base material. A shape of the base may be a plate shape so that the noble metal thin film may be uniformly and flatly formed. As an example of the board, an amorphous board including glass, an oxide board, a nitride board, a semiconductor board, or a multi-layered board thereof may be used. Here, the oxide board, the nitride board or the semiconductor board may be polycrystalline, monocrystalline, or amorphous.

As the noble metal thin film formed on the base serves to be bonded to the peptide to fix the peptide to the base and amplify a secondary ion mass signal of the peptide (including peptide itself and a peptide specifically reacted with the disease marker), it is preferable that the noble metal thin film is a gold thin film spontaneously bonded to an element contained in amino acid configuring a terminal of the peptide and effective amplifying the secondary ion mass signal of the peptide.

As shown in FIG. 1A, a noble metal thin film 200 is formed on one surface of a base 100, wherein the noble metal thin film 200 formed on the base 100 may include polygonal, oval or circular noble metal thin films 201 to 203.

As shown in FIG. 1B, the noble metal thin film 200 may include a plurality of noble metal thin films 201 to 203 formed on the surface of the base 100 in a state in which they are spaced apart from each other to thereby be separated.

In addition, as shown in FIG. 1C, the base may include a base 100′ in which at least one concave groove formed therein, wherein bottom surfaces of the concave grooves may be provided with the noble metal films 201 to 203, and a predetermined step may be formed between the noble metal films 201 to 203 and the surface of the base.

A side (a surface except for the bottom surface) of the concave groove may be an inclined side (tapered side) at a predetermined angle in order to smoothly detect secondary ion materials at the time of SIMS analysis. In the case in which the side of the concave groove is inclined, it is preferable that the side is inclined so as to become narrow toward the bottom surface of the concave groove. The bottom surface of the concave groove may have polygonal, oval, or circular shape.

The disease marker detection kit according to the present invention may include the first storage unit filled with the reactant containing the peptide specifically reacting with the disease marker and the second storage unit filled with the test substance containing the biological sample of the possible disease carrier.

The peptide of the reactant filled in the first storage unit contacts the biological sample of the test substance filled in the second storage unit to be mixed with each other, and in the case in which the disease marker is contained in the biological sample, the peptide specifically reacts with the disease marker, such that a specific reactant that is a peptide specifically reacted with the disease marker is formed.

More specifically, in the disease marker detection kit, in order to detect the disease marker used to determine presence or absence of the disease in the possible disease carrier, using the fact that the secondary ion mass signal of the peptide may be effectively amplified by the gold thin film, after the disease marker reacts with the peptide and the peptide reacted with the disease marker (the specific reactant) is self-assembled on the gold thin film, the secondary ion mass signal of the peptide reacted with the disease marker is amplified using the gold thin film. Therefore, a kind and content of the disease marker may be sensitively, precisely, and reproducibly measured using a position, intensity, or position and intensity of a secondary ion mass spectrum of the peptide (specific reactant) changed by the specific reaction with the disease marker.

In describing the present invention, the content of the disease marker may be measured in the case in which the disease marker is not detected, and in this case, the content is indicated as “0”.

The reactant may be preferably a solution in which the peptide specifically reacting with the disease marker is dispersed and diluted at a predetermined concentration, and in order to simultaneously detect two or more different disease markers, the reactant may include two or more different peptides having specificity to each of the disease markers, for each of the disease markers. In this case, the two or more different peptides contained in the reactant may have predetermined concentrations different from each other.

As the solution in which the peptide is dispersed, a sterilized liquid material that is stable for a long period of time and does not generate biochemical changes in the biological sample may be used. For example, phosphate buffered saline, triple distilled water, or dimethyl sulfoxide (DMSO) may be used.

The test substance is a solution containing biological samples extracted or extracted from the possible disease carrier to be treated, wherein the biological sample may be feces, urine, tears, saliva, external secretions of the skin, external secretions of a respiratory tract, external secretions of enteric track, external secretions of the digestive tract, plasma, serum, blood, spinal fluid, lymph fluid, body fluids, tissue, tissue homogenates, part of tissue, cells, cell extracts, or in vitro cell culture.

The test substance may also be a solution in which the biological sample extracted and treated according to a predetermined protocol is dispersed and diluted at a predetermined concentration, similarly to the reactant. As the solution in which the biological sample is dispersed, a sterilized liquid material that is stable for a long period of time and does not generate biochemical changes in the biological sample may be used. For example, phosphate buffered saline, or triple distilled water may be used.

The first storage unit may receive the reactant and be configured to include a container part provided with an inlet into which the reactant is introduced and a cap part connected to the inlet of the container part to seal the reactant from the outside. In this case, the container part may be preferably sealed except for the inlet.

The container part may be made of any material as long as the material may be easily processed in a container shape in which a liquid material may be received, not generate impurities and biochemical changes in the reactant, and physically and chemically block the reactant from the outside of the container part. For example, the container part may be made of glass or plastic.

The cap part is coupled to the inlet of the container part to seal the reactant filled in the container part from the outside. The coupling between the cap part and the inlet may include physical coupling, chemical coupling including the coupling by an adhesive, or dissolution coupling.

A material of the cap part may also be any material as long as the material has porosity, does not generate biochemical changes in the reactant, and effectively, physically, and chemically block the reactant from the outside, but the material may be preferably glass or plastic.

The second storage unit may receive the test substance, similarly to the first storage unit and be configured to include a container part provided with an inlet into which the test substance is introduced and a cap part connected to the inlet of the container part to seal the test substance from the outside. In this case, the container part may be preferably sealed except for the inlet.

Similarly to the container part of the first storage unit, the container part of the second storage unit may be made of any material as long as the material may be easily processed in a container shape in which a liquid material may be received, not generate impurities and biochemical changes in the test substance, and block the test substance from the outside of the container part. For example, the container part may be made of plastic.

Similarly to the cap part of the first storage unit, the cap part of the second storage unit is coupled to the inlet of the container part to seal the test substance filled in the container part from the outside. The coupling between the cap part and the inlet may include physical coupling, chemical coupling including the coupling by the adhesive, or dissolution coupling.

A material of the cap part may also be any material as long as the material has porosity, does not generate biochemical changes in the test substance, and effectively, physically, and chemically block the test substance from the outside, but the material may be preferably plastic.

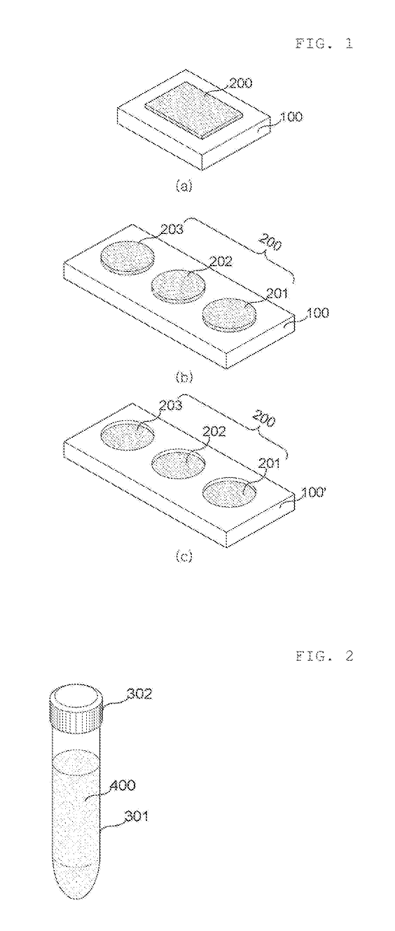

FIG. 2 is a view showing an example of a first storage unit configured to include a container part 301 and a cap part 302 and including a reactant 400 sealed stored therein. Although not shown, the second storage unit may also have a shape similar to that in FIG. 2.

The disease detection kit according to the present invention may further include a mixing unit mixing the reactant and the test substance with each other to prepare a detection substance containing the specific reactant, which is the peptide specifically reacted with the disease marker contained in the biological sample and a contact unit contacting the detection substance prepared by the mixing unit with the base to bond the specific reactant to the noble metal film of the base.

The reactant filled in the first storage unit and the test substance filled in the second storage unit are mixed by the mixing unit, and the detection substance containing the specific reactant may be prepared by the mixing unit.

Preferably, the mixing unit may be configured to include a sealing container part sealing and storing the reactant introduced from the first storage unit and the test substance introduced from the second storage unit and a stirring part applying vibration or rotation to the sealing container part. In this case, the mixing unit may further include a constant temperature part maintaining a temperature of the sealing container part to be constant.

The sealing container part may be similar to the first storage unit (or the second storage unit) of FIG. 2, and the stirring part may be a stirrer or a mixer.

The detection substance prepared by the mixing unit may be contacted with the base by the contact unit, and the specific reactant contained in the detection substance may be bonded to the noble metal thin film of the base.

Here, in the case in which peptides that do not react with the disease marker are present in the detection substance, the peptide (unreacted peptides) as well as the specific reactant may be bonded to surface atoms of the noble metal thin film.

The detection substance prepared by the mixing unit is contacted with the noble metal thin film of the board by the contact unit, such that at least one material selected among the specific reactant and the peptides (unreacted peptides) contained in the detection substance may be bonded to the noble metal thin film to thereby be fixed to the base.

The contact unit is a unit storing the detection substance and allowing the board to be immersed into the detection material, extracting a predetermined amount of detection substance from the mixing unit to drop the extracted substance onto the board, or allowing the detection substance to flow on a surface of the board at a predetermined rate. In addition, the contact unit may further include a unit applying vibration at the time of contact of the detection substance and the board and a temperature maintaining unit maintaining temperatures of the detection substance and the board contacting the detection substance. As an example of the contact unit, there are a simple pipette and a micro channel coupled to the board and sealed in which the detection substance flows.

The disease detection kit according to the present invention may further include a storage part storing a test spectrum, which is a secondary ion mass spectrum of a test sample in which the specific reactant of the detection substance is bonded to the noble metal thin film; and a determining part determining a kind and a content of the disease marker contained in the biological sample based on at least one factor selected from the secondary ion mass and intensity of a peak present in the test spectrum stored in the storage part.

Preferably, the storage part may further store a reference spectrum, which is a secondary ion mass spectrum of a reference sample in which the reactant itself contacts the noble metal thin film of the base, such that the peptide (unreacted peptide) of the reactant is bonded to the noble metal thin film.

In addition, each of the reference spectra may be stored in the storage part for each kind and content of the peptides contained in the reactant of the first storage unit, and the kind and content information of the biological sample contained in the test substance of the second storage unit may be also stored therein. Further, in the case in which detection substance prepared by the mixing unit is partially contacted to the board including the noble metal thin film formed thereon by the contact unit, volume information of the detection substance contacted with the board may also be stored therein.

In the disease detection kit according to the present invention, the disease marker may be a marker for cancer and be cell wall lytic enzymes.

More specifically, the cell wall lytic enzyme may be at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23, and a reference for determining presence or absence of the disease in the possible disease carrier of which a biological sample is extracted is prepared through at least one factor selected from the kinds and contents of MMPs contained in the biological sample.

Here, MMPs spectra, which are secondary ion mass spectra measured after mixing MMPs solutions (solutions in which at least one MMP are dispersed at a predetermined concentration) prepared for each kind and content of the above-MMPs with the reactant and contacting the prepared MMPs solutions with the noble metal thin film, may be further stored as another reference spectra.

Further, analysis parameter information including information on a material of an ion beam for SIMS, energy of the ion beam, mass spectroscopy conditions including a mass spectroscopy mode and an analysis area, the kinds and the contents of the peptides contained in the reactant, may be further stored in the storage part. In the case in which the SIMS is performed in order to obtain the reference spectrum and the test spectrum, the same analysis parameter should be maintained.

Furthermore, information obtained by measuring the secondary ion mass intensity of the peptide specifically reacted with the disease marker for each variable through designed experiments using a kind of disease markers, the concentration of the disease marker contained in the reactant, a kind of peptides specifically reacting with the disease marker, and the concentration of the peptide contained in the reactant as variables and tabulating the measured results in a look up table form may be further stored in the storage part. The look up table may be used to determine the kind and the content of the disease marker that is performed by the determining part.

As an example of the storage part, there is a computer readable storage medium such as a RAM, a ROM, a CD, a magnetic recording medium.

The determining part may determine a kind and a content of the disease marker contained in the biological sample based on at least one factor selected from the secondary ion mass and intensity of a peak present in the test spectrum stored in the storage part to provide the reference for determining presence or absence of the disease in the possible disease carrier of which the biological sample is extracted.

Preferably, the determining part may load the reference spectrum of the reactant used in order to obtain the test spectrum, interlocking with the storage part, and compare the secondary ion mass and intensity of the secondary ion mass peak of the reference spectrum with the secondary ion mass and intensity of the secondary ion mass peak of the test spectrum with each other.

Through the comparison as described above, a secondary ion mass corresponding to a peak disappearing or reduced in the reference spectrum and a secondary ion mass corresponding to a peak generated in the test spectrum are extracted, and the disease marker specifically reacted with the peptide is estimated, thereby deriving a kind of disease marker.

In addition, through the comparison, a content of the disease marker contained in the test substance may be derived from the secondary ion intensity of the peak disappearing or reduced in the reference spectrum and the secondary ion intensity of the peak generated in the test spectrum.

In this case, the kind and the content of the disease marker derived in the determining part may become a standard for determining the presence or absence of the disease in the possible disease carrier in themselves, and the presence or absence of the disease itself may be determined by comparing the kind and the content (measured value) of the disease marker derived in the determining part with content (external inputted value) information of the disease marker for each disease marker stored in the storage part and used as the standard for determining the disease of the possible disease carrier.

As described above, the disease marker detection kit may be a detection kit detecting a marker for cancer, and the disease marker may be a cell wall lytic enzyme.

More specifically, the cell wall lytic enzyme may be at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

The MMP specifically reacts with a peptide having a mass in a range of 800 to 1300 Da to cut a predetermined site of a substrate, such that a secondary ion mass of the cut substrate may be effectively detected due to matrix action of the noble metal thin film, thereby making it possible to effectively measure an activity of the MMP. In this detection method, the detection method may be simplified as compared to the existing method, detection may be performed in a significantly short time, and measurement may be accurately and reproducibly performed. As the MMPs are highly expressed in cancer cells and cancer tissue of almost every patient, measurement of an amount of MMPs in tissue or body fluids may be sufficiently used as a diagnostic marker for cancer. In the case of breast cancer, large amounts of MMPs-2 and -9 are detected in the blood fluids and the tissue such as urine or serum, and in the case of bladder cancer, MMP-9 is detected in the brain, and MMPs-2 and -9 are detected in urine (Roy, R. 2009, J. Clin. Oncol., 5287-5297).

More particularly, the MMP contained in the test substance specifically reacts with the peptides contained in the reactant, such that a specific site is cut according to the MMP substrate specificity, and both of the cut substrate and the original substrate that is not cut are self-assembled on the noble metal thin film of the board to thereby be bonded to the noble metal thin film.

When an ion beam for the SIMS is irradiated, secondary ion mass spectra of two peptides having different masses from each other according to the presence or absence of the MMPs may be easily and sensitively measured due to signal enhancement effect of peptides by the noble metal thin film.

In the disease detection kit, in order to specifically react with the above-mentioned cell wall lytic enzymes, the peptide may have a specific amino acid site for each lytic enzyme and be an unlabeled substrate. For example, in the case of MMPs-2 and -9, the MMPs-2 and -9 may specifically recognize and dissolve Gly-Val, Gly-Leu, Gly-Gly, Gly-Asn, and Gly-Ser among amino acid sequences.

In order to allow the peptide specifically reacted with the cell wall lytic enzymes to be uniformly bonded to the noble metal thin film of the board as a monolayer, it is preferable that the peptide may be a peptide in which an amino acid in an amine or carboxyl terminal site is cysteine. In addition, in order to allow the peptide of the specific reactant to be spontaneously bonded (self-assembled) to the noble metal thin film and effectively increase efficiency of the SIMS of the bonded specific reactant, it is preferable that the noble metal thin film is a gold thin film.

As described above, the biological sample contained in the test substance may be feces, urine, tears, saliva, external secretions of the skin, external secretions of a respiratory tract, external secretions of enteric track, external secretions of the digestive tract, plasma, serum, blood, spinal fluid, lymph fluid, body fluid, tissue, tissue homogenates, part of tissue, cells, cell extracts, or in vitro cell culture.

Here, the disease marker is the marker for cancer, and in the case in which the disease marker is the cell wall lytic enzyme, the biological sample may be urine, plasma, serum, or pancreatic fluid.

Hereinafter, a detection method of a disease marker will be described in detail.

The detection method for a disease marker according to the present invention, which is a method of detecting a content of a disease marker in order to provide information required for diagnosis of the presence or absence of the disease in a possible disease carrier, may include: a) mixing a reactant containing a peptide specifically reacting with the disease marker with a test substance containing a biological sample of the possible disease carrier to prepare a detection substance containing a specific reactant, which is a peptide specifically reacted with the disease marker contained in the biological sample; b) contacting the detection material with the base including a noble metal thin film formed thereon to bond unreacted peptide and the specific reactant to the noble metal thin film; c) performing a SIMS on the base contacted with the detection substance to obtain a test spectrum; and d) detecting a kind and a content of the disease marker based on at least one factor selected from a secondary ion mass and intensity of a peak present in the test spectrum.

In this case, before performing the step a), a′) contacting the reactant itself containing the peptide specifically reacting with the disease marker with the base including the noble metal thin film formed thereon to allow the peptide contained in the reactant to be self-bonded to the noble metal thin film and performing the SIMS on the self-bonded peptide to obtain a reference spectrum, which is a secondary ion mass spectrum of the peptide, may be further performed.

The peptide may be a non-labeled substrate having an amino acid site specifically reacting with the disease marker and include an unlabeled substrate having amino acid sites specifically reacting with at least two different disease markers, respectively.

Before performing step a), a pre-treatment step of precipitating the disease marker contained in the biological sample using an immunoprecipitation method may be preferably performed, independently of step a′), and in this case, the test substance may be preferably a precipitation substance precipitated by the immunoprecipitation method or a solution containing the precipitate substance.

In detail, the precipitation substance precipitated by the immunoprecipitation method is separated from the supernatant and recovered, and then the precipitation substance is mixed with the peptide specifically reacting with the disease marker, such that the detection substance containing the reactant, which is the peptide reacted with the disease marker contained in the biological sample may be preferably prepared.

Here, the test substance may be prepared by dispersing the separated and recovered precipitation substance in a liquid material, wherein as the liquid material, a sterilized liquid material that is stable for a long period of time and does not generate biochemical changes in the biological sample may be used. For example, phosphate buffered saline, or triple distilled water may be used.

In the case in which the biological sample (for example, serum) includes other biological substances (for example, protein) in plural as well as the disease marker to be desired, or in the case in which the peptide has amino acid sites specifically reacting with at least two different disease markers, respectively, in order to improve the bonding with the noble metal thin film and/or accuracy of the measurement, the pre-treatment step may be performed.

The reactant in step a) or step a′) may be a solution in which the peptide specifically reacting with the disease marker is dispersed and diluted at a predetermined concentration, and in order to simultaneously detect two or more different disease markers, the reactant may include two or more different peptides having specificity to each of the disease markers, for each of the disease markers or a single peptide having specificity to both of the two different markers. Here, in the case in which the reactant contains two or more different peptides, the peptides may have different concentrations.

As the solution in which the peptide is dispersed, a sterilized liquid material that is stable for a long period of time and does not generate biochemical changes in the biological sample may be used. For example, phosphate buffered saline, triple distilled water, or dimethyl sulfoxide (DMSO) may be used.

In order to easily prepare the specific reactant by being mixed with the test substance in step a) and obtain reproducible and reliable mass marker detection results by forming a self-bonded film on the noble metal thin film at the time of contacting the board including the noble metal thin film in step b), it is preferable that the peptide of the reactant has a sufficient concentration at which the peptide may form a monolayer self-bonded film on the noble metal thin film and has a content higher than the maximum content of the disease marker contained in the biological sample. As an example, the reactant may contain 1 to 30 μmol concentration of peptide.

The test substance in step a) is the solution containing the biological sample extracted or extracted from the possible disease carrier to be treated (pre-treated), wherein the biological sample may be feces, urine, tears, saliva, external secretions of the skin, external secretions of a respiratory tract, external secretions of enteric track, external secretions of the digestive tract, plasma, serum, blood, spinal fluid, lymph fluid, body fluid, tissue, tissue homogenates, part of tissue, cells, cell extracts, or in vitro cell culture.

Here, in the case in which the biological sample is a liquid such as urine, the biological sample itself may be used as the test substance, and in the case in which the test substance is a pre-treated biological sample, the pre-treatment may include treatment by the above-mentioned immunoprecipitation method.

When a solution in which the biological sample is dispersed is used as the test substance, a solution in which the biological sample extracted and treated according to a predetermined protocol is inputted into and mixed with a liquid material at a predetermined volume or weight may be used as the test substance. As liquid material of the test substance, a sterilized liquid material that is stable for a long period of time and does not generate biochemical changes in the biological sample may be used. For example, phosphate buffered saline, or triple distilled water may be used.

In this case, mixing different reactants and test substances are mixed with each other, thereby making it possible to prepare two or more detection substances prepared in step a). As an example, a first reactant and a first test substance may be mixed to thereby prepare a first detection substance and a second reactant independent from the first reactant and a second test substance independent from the first test substance may be mixed to thereby prepare a second detection substance.

At the time of preparing the detection substance in step a), the reactant and the test substance are mixed and reacted with each other at a predetermined temperature (as an example, 20 to 40° C., and as a substantial example, 37° C.).

In the case in which two or more detection substances different from each other are prepared in step a), each of the detection substances may be applied to a different region of the noble metal thin film, and specific reactants different from each other may be self-assembled on each of the regions of the noble metal thin film. To this end, as described above, the board on which the noble metal thin film is formed as shown in FIGS. 1B and 1C.

Particularly, the detection method of a disease marker according to the present invention is a detection method for a marker for cancer, wherein the disease marker may be a cell wall lytic enzyme. More specifically, the cell wall lytic enzyme may be at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

In the case in which the disease marker is the cell wall lytic enzyme, the biological sample may be urine, plasma, serum, or pancreatic fluid.

In this case, in order to specifically react with the above-mentioned cell wall lytic enzyme, the peptide may have a specific amino acid site for each lytic enzyme and be the unlabeled substrate.

Here, the peptide contained in the reactant may include an unlabeled substrate having an amino acid site specifically reacting with all of the two or more cell wall lytic enzymes different from each other.

In order to allow the peptide specifically reacted with the cell wall lytic enzymes to be maximally uniformly bonded to the noble metal thin film of the board as a monolayer, it is preferable that the peptide may be a peptide in which an amino acid in an amine or carboxyl terminal site is cysteine.

The contact in step b) may include impregnation of the board into the detection substance, a drop of the detection substance onto the board on which the noble metal thin film is formed, or a flow of the detection substance onto the board on which the noble metal thin film is formed at a predetermined rate.

In step d), the detecting of a kind and a content of the disease marker based on at least one factor selected from a secondary ion mass and intensity of a peak present in the test spectrum is performed. It is preferable that step d) is performed based on changes in positions and intensities between the secondary ion mass peaks of the reference spectrum obtained in step a′) and the test spectrum.

In detail, step d) may include d1) comparing the secondary ion mass and intensity of secondary ion mass peaks of the reference spectrum and the secondary ion mass and intensity of the secondary ion mass peaks of the test spectrum; and d2) extracting a secondary ion mass corresponding to a peak disappearing or reduced in the reference spectrum and a secondary ion mass corresponding to a peak generated in the test spectrum to derive a kind of disease marker specifically reacted with the peptide and extracting a secondary ion intensity corresponding to the peak disappearing or reduced in the reference spectrum and a secondary ion intensity corresponding to the peak generated in the test spectrum to derive the content of the disease marker contained in the test substance.

In this case, the kind and the content of the disease marker derived in step d) may become a standard for determining the presence or absence of the disease in the possible disease carrier in themselves, and the presence or absence of the disease itself may be determined by comparing the kind and the content (measured value) of the disease marker derived in step d) with the kind of preset disease markers and critical secondary ion mass intensity of the corresponding disease marker.

In addition, through designed experiments, using the kind of disease markers, the concentration of the disease marker contained in the reactant, the kind of peptides specifically reacting with the disease marker, and the concentration of the peptide contained in the reactant as variables, the secondary ion mass intensity of the peptide specifically reacted with the disease marker is measured for each variable and then the measured results are tabulated in a look up table form, such that the kind and the concentration of the disease marker contained in the biological sample to be actually measured may be derived.

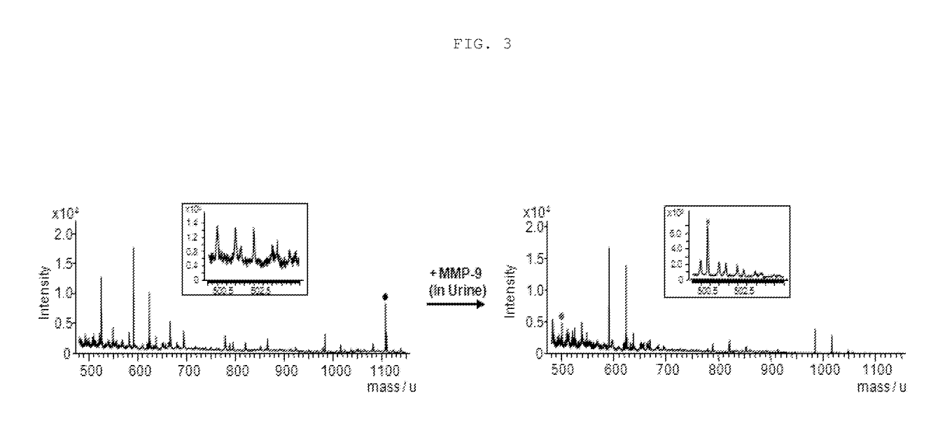

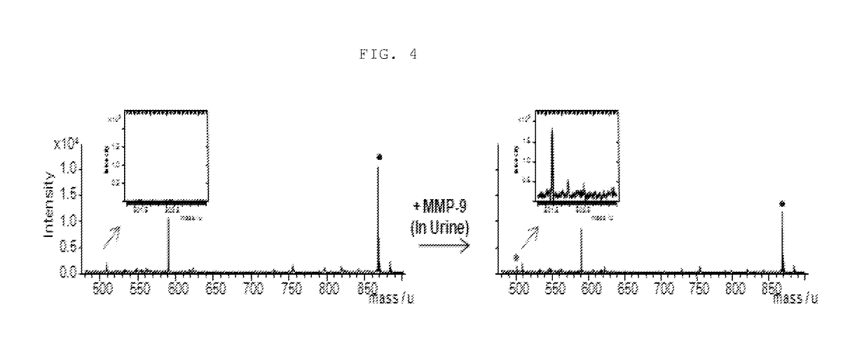

FIG. 3 is a view showing a secondary ion mass spectrum (reference spectrum, a left graph in FIG. 3) measured after contacting a peptide with a base on which a gold thin film is formed and a secondary ion mass spectrum (test spectrum, a right graph in FIG. 3) measured after contacting the peptide mixed with urine with the base on which the gold thin film is formed, using a sample in which MMP-9 is loaded in urine (250 μL) obtained from a normal person as a biological sample and using Ac-KGPRQITAGGGC (acetylation-KGPRQITAGGGC) as a peptide specifically reacting with a disease marker.

More specifically, 25 μM of Ac-KGPRQITAGGGC, which is a MMP-9 specific substrate, was added to the urine containing MMP-9, and reaction was carried out at 37° C. for 1 hour. A bond between Gln and Ile in a peptide sequence was broken by activity of the MMP-9, and a specific reactant (mass: 500.5), which is a peptide formed after reaction with the existing peptide (mass: 1109.5), was formed according to the activity of MMP-9. After this reactant was bonded to the gold thin film board for 30 minutes, in order to perform measurement on only the peptides bonded to the gold thin film, the gold thin film board was washed with triple distilled water, and then the SIMS was performed using a time-of-flight secondary ion mass spectrometry (TOF-SIMS).

As seen in FIG. 3, it could be confirmed that the secondary ion mass of the cell wall lytic enzyme contained in urine may be effectively detected by the gold thin film and peptide, Ac-KGPRQITAGGGC, and it could be appreciated that activated gelatinase B (MMP-9) is present in urine through the positions and intensities of the secondary ion mass peaks of the reference spectrum and the test spectrum.

FIG. 4 is a view showing a secondary ion mass spectrum (reference spectrum, a left graph in FIG. 4) measured after contacting a peptide with a base on which a gold thin film is formed and a secondary ion mass spectrum (test spectrum, a right graph in FIG. 4) measured after contacting the peptide mixed with precipitation substance separated from the urine by the immunoprecipitation method with the base on which the gold thin film is formed, using a sample in which MMP-9 is loaded in urine (250 μL) obtained from a normal person as a biological sample and using Ac-GPLGMRGLC (acetylation-GPLGMRGLC), which is a general peptide of the cell wall lytic enzyme, as a substrate of MMP-9.

In detail, the pre-treatment by the immunoprecipitation method was performed as follows. A specific antigen of MMP-9 was added to urine, and reaction was carried out for 2 hours to prepare an immune-complex, and then the immune-complex was precipitated by adding protein G agarose. 2 μM of Ac-GPLGMRGLC, which is the substrate of the MMP-9, was added to the precipitated agarose (immune complex), and reaction was carried out at 37° C. for 1 hour. A bond between Gly and Met in a peptide sequence was broken by activity of the existing MMP-9 at the time of reaction. Therefore, a specific reactant (mass: 501.4), which is a peptide formed after reaction with the residual peptide (mass: 867.7) in an unreacted state, was formed according to the activity of MMP-9. After this reactant was applied and bonded to the gold thin film board for 30 minutes, in order to perform measurement on only the peptide bonded to the gold thin film, the gold thin film board was washed with triple distilled water, and then TOF-SIMS was performed.

As set forth above, the detection kit according to the present invent may non-invasively detect the kind and the content of the disease marker used to determine the presence or absence of the disease of the possible disease carrier without labeling; simply, rapidly, accurately, and reproducibly detect the content of the disease marker; and be mass-produced with low cost.

With the detection method according to the present invention, the kind and the content of the disease marker used to determine the presence or absence of the disease of the possible disease carrier may be non-invasively detected without labeling, and the kind and the content of the disease marker may be simply, accurately, and reproducibly detected in a short time.

Hereinabove, although the present invention is described by specific matters, exemplary embodiments, and drawings, they are provided only for assisting in the entire understanding of the present invention. Therefore, the present invention is not limited to the exemplary embodiments. Various modifications and changes may be made by those skilled in the art to which the present invention pertains from this description.

Therefore, the spirit of the present invention should not be limited to the above-described exemplary embodiments, and the following claims as well as all modified equally or equivalently to the claims are intended to fall within the scope and spirit of the invention.

1. A disease detection kit used together with a secondary ion mass spectrometer in order to detect a disease marker contained in a biological sample, the detection kit comprising:
a base including a noble metal thin film formed thereon;
a reactant containing peptide specifically reacting with the disease marker;
a first storage unit filled with the reactant;
a test substance containing a biological sample of a possible disease carrier; and
a second storage unit filled with the test substance.

2. The detection kit of claim 1, further comprising:
a mixing unit mixing the reactant and the test substance with each other to prepare a detection substance containing a specific reactant, which is the peptide specifically reacted with the disease marker contained in the biological sample; and
a contact unit contacting the detection substance prepared by the mixing unit with the base to thereby bond the specific reactant to the noble metal film of the base.

3. The detection kit of claim 2, further comprising
a storage part storing a test spectrum, which is a secondary ion mass spectrum of a test sample in which the specific reactant of the detection substance is bonded to the noble metal thin film; and
a determining part determining a kind and a content of the disease marker contained in the biological sample based on at least one factor selected from the secondary ion mass and intensity of a peak present in the test spectrum stored in the storage part.

4. The detection kit of claim 1, wherein the disease marker is a marker for cancer and a cell wall lytic enzyme.

5. The detection kit of claim 4, wherein the cell wall lytic enzyme is at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

6. The detection kit of claim 5, wherein the peptide has a specific amino acid site for the cell wall lytic enzyme and is an unlabeled substrate.

7. The detection kit of claim 6, wherein an amino acid of an amine terminal or a carboxyl terminal of the peptide is cysteine.

8. The detection kit of claim 1, wherein the noble metal thin film is a gold thin film.

9. The detection kit of claim 1, wherein the biological sample is feces, urine, tears, saliva, external secretions of the skin, external secretions of a respiratory tract, external secretions of enteric track, external secretions of the digestive tract, plasma, serum, blood, spinal fluid, lymph fluid, body fluids, tissue, tissue homogenates, part of tissue, cells, cell extracts, or in vitro cell culture.

10. A detection method of a disease marker in order to provide information required in diagnosis of a disease, the detection method of a disease marker comprising:
a) mixing a reactant containing a peptide specifically reacting with the disease marker with a test substance containing a biological sample of the possible disease carrier to prepare a detection substance containing a specific reactant, which is a peptide specifically reacted with the disease marker contained in the biological sample;
b) contacting the detection material with the base including a noble metal thin film formed thereon to bond unreacted peptide and the specific reactant to the noble metal thin film;
c) performing a secondary ion mass spectroscopy (SIMS) on the base contacted with the detection substance to obtain a test spectrum; and
d) detecting a kind and a content of the disease marker based on at least one factor selected from a secondary ion mass and intensity of a peak present in the test spectrum.

11. The detection method of a disease marker of claim 10, further comprising precipitating the disease marker contained in the biological sample using an immunoprecipitation method before step a),
wherein the test substance includes a precipitation substance precipitated by the immunoprecipitation method.

12. The detection method of a disease marker of claim 10, wherein the disease marker is a marker for cancer and a cell wall lytic enzyme.

13. The detection method of a disease marker of claim 12, wherein the cell wall lytic enzyme is at least one matrix metalloproteinase (MMP) selected from collagenase 1, gelatinase A, stromelysin 1, matrilysin, collagenase 2, gelatinase B, stromelysin 2, stromelysin 3, macrophage elastase, collagenase 3, MT1-MMP, MT2-MMP, MT3-MMP, MT4-MMP, collagenase 4, MMP-19, enamelysin, XMMP, CMMP, and MMP-23.

14. The detection method of a disease marker of claim 13, wherein the peptide has an amino acid site specifically reacting with the cell wall lytic enzyme and is an unlabeled substrate.

15. The detection method of a disease marker of claim 12, wherein the peptide has an amino acid site specifically reacting with two or more cell wall lytic enzymes different from each other and is an unlabeled substrate.

16. The detection method of a disease marker of claim 12, wherein an amino acid of an amine terminal or a carboxyl terminal of the peptide is cysteine.

17. The detection method of a disease marker of claim 10, wherein the noble metal thin film is a gold thin film.

18. The detection method of a disease marker of claim 12, wherein the biological sample is feces, urine, tears, saliva, external secretions of the skin, external secretions of a respiratory tract, external secretions of enteric track, external secretions of the digestive tract, plasma, serum, blood, spinal fluid, lymph fluid, body fluids, tissue, tissue homogenates, part of tissue, cells, cell extracts, or in vitro cell culture.
